(19) United States
(12) Patent Application Publication
(10) Pub. No.: US 2025/0152219 A1
(43) Pub. Date: May 15, 2025

(54) BONE FIXING IMPLANT DEVICES AND SYSTEMS

(71) Applicant: PBC INNOVATIONS LIMITED, Shannon, Co. Clare (IE)

(72) Inventors: Robert A. FLAVIN, Shannon (IE); Ivana IVANKOVIC, Shannon (IE)

(21) Appl. No.: 18/838,521

(22) PCT Filed: Feb. 16, 2023
(86) PCT No.: PCT/EP2023/053896

§ 371 Date: Aug. 14, 2024

(30) Foreign Application Priority Data
Feb. 17, 2022 (GB) 2202154.7

(51) Int. Cl. A61B 17/86; A61B 17/00; A61B 17/56; A61B 17/68; A61B 17/88

(52) U.S. Cl. CPC A61B 17/863; A61B 17/864; A61B 17/8875; A61B 17/8897; A61B 2017/00482; A61B 2017/564; A61B 2017/681; A61B 2017/8655

(57) ABSTRACT

The present specification relates to devices, systems and methods for fixing a fracture in orthopaedic procedures. The specification provides a bone implant for supporting a damaged bone or for fixing a bone fracture, comprising: a shaft having a proximal end and a distal end and a longitudinal axis (L-L); the shaft having a threaded shaft portion extending in a proximal to distal direction and located proximate the distal end; the shaft having an unthreaded shaft portion located between a proximal end of the shaft and the threaded shaft portion; an archway extending from the distal end of the threaded shaft portion, the archway forming a tunnel that is configured to receive a bone fixing screw in a direction (T-T) that is inclined to the longitudinal axis; and the proximal end of the shaft configured to allow a user to drive the shaft into bone. Related methods and systems are also described.

### CROSS-REFERENCE TO RELATED APPLICATIONS

This application is a U.S. National Stage of International Patent Application No. PCT/EP2023/053896 filed Feb. 16, 2023, which claims priority to United Kingdom Patent Application No. 2202154.7 filed Feb. 17, 2022, both of which are incorporated by reference herein as if reproduced in their entireties.

### FIELD

The present application relates to a bone fixing implant particularly suited for supporting damaged bones including for example ankles. The implant is optimized to increase support, reduce the risk of infection, increase early weight bearing and is of particular use in surgery to repair bone injuries and fractures. The present application claims priority to patent application GB2202154.7 filed on Feb. 17 2022, the contents of which are herein incorporated by reference.

### BACKGROUND

Bone fracture injuries are common and it is important such injuries heal as robustly and quickly as possible. As an example of a bone fracture injury, the ankle is the most commonly injured major joint in the body, and a common injury is fracture of the medial malleolar. It is particularly important that bone fracture injuries heal as robustly and quickly as possible. Surgery can help to support and align a damaged bone to assist healing. Known solutions include fixing with one or more fixations such as screws and/or plates. Unfortunately, known fixations are rarely as secure to one would like and have a tendency to work loose or pull-out. Relatedly, the use of known fixations is associated with high complication rates e.g. infections and high levels of reported pain following surgery. There is also a risk of multiple or repeat surgical operations being required, and often there is often a prolonged period after surgery in which repaired ankles have reduced weight-bearing ability. These risks are increased for older patients because as humans age, their bone quality decreases. Low-quality bone is bone that is not well-suited for securing to or from. There are many reasons bone might be low-quality such as osteoporosis or other disease, trauma or other disruption, or the target site comprising cancellous/trabecular (spongy) bone as opposed to cortical (hard) bone. Therefore, bone surgery in older patients, whilst often required, is especially challenging.

Partly due to the above-mentioned challenges, non-surgical approaches are often used. However, such approaches can result in arthritis and severe long-term discomfort. Ultimately, such approaches may even result in a need to replace a damaged bone for example an ankle i.e. to perform total ankle replacement surgery. As these risks grow over time, these aspects are particularly relevant for surgery on younger patients.

Consequently, there is a need for an improved fixation that will address at least some of these problems.

### SUMMARY

Bone fixing implants are described that are particularly suitable for use in medial-malleolar fixing surgery. The implants provide a very secure hold and are well-suited for use in low-quality bone. The implants have a low failure rate relative to known fixations, and provide a secure hold that decrease the likelihood of other adverse effects. The secure hold of the implants means they can often be used instead of plates. Consequently, much smaller skin incisions are required improving infection control and reducing surgical trauma.

The implant of the arrangements of the specification uses one or more interlocking substantially perpendicular screws to maximise the stability of the implant. The implant is in a sense suspended from the one or more substantially perpendicular screws. This suspension effect has been found to significantly increase the implants ability to resist being pulled out. In some embodiments, the pullout strength was approximately an order of magnitude (250N) greater than standard bone fixing screw.

In some embodiments, the implant supports two interlocking screws, one through the distal end to securely suspend the implant and another at approximately the longitudinal midpoint to further compress the bone. This is particularly advantageous for complex fractures including for example medial malleolar fractures that may otherwise require invasive plates and/or a lot of screws. The implant may be designed having different diameters for different applications and to ensure tunnels through the implant that support the screws are located in strong sections whilst minimizing the weight and size of the implant.

The implants may have a wide head with small anchors or ‘spikes’ that cusp the malleolus when the implant is inserted. These spikes also ensure extra stability and further decrease the chances of the implant being pulled or twisting out.

The implants may also have a thread with an asymmetric profile, which is less steep on the proximal side than on the distal side. Therefore, a surgeon can easily pull, or twist, out the anchor and reposition the implant if necessary. Due to the asymmetric profile, the twisted retraction of the implant is likely to cause little damage to the engaged bone and is less likely to damage the thread path that the threads cut in the engaged bone during insertion.

According to a first aspect there is provided a bone implant for supporting a damaged bone or for fixing a bone fracture, comprising:
a shaft having a proximal end and a distal end and a longitudinal axis (L-L);
the shaft having a threaded shaft portion extending in a proximal to distal direction and located proximate the distal end;
   the shaft having an unthreaded shaft portion located between a proximal end of the shaft and the threaded shaft portion;
an archway extending from the distal end of the threaded shaft portion, the archway forming a tunnel that is configured to receive a bone fixing screw in a direction (T-T) that is inclined to the longitudinal axis; and
   the proximal end of the shaft configured to allow a user to drive the shaft into bone.

In one embodiment of the first aspect, the bone implant further comprising a connector at the proximal end of the shaft configured for coupling to a driving tool to drive or twist the implant into bone. The direction (T-T-transverse axis) may be generally perpendicular to the longitudinal axis. The shaft may further comprise a second tunnel extending across one of the threaded or the unthreaded shaft in a second direction (I-I) that is inclined to the longitudinal axis. The bone fixation implant may be a cannulated bone fixation implant and comprise a lumen in the longitudinal direction for coupling to a guidewire.

The unthreaded shaft may comprise a proximal part and a distal part. The second tunnel may be disposed in the distal part. The second tunnel may be disposed between the proximal part and the distal part. The driver may comprise two or more wings, the two or more wings extending in a radial direction away from the longitudinal axis and symmetrically arranged about the longitudinal axis. The radial tip of each wing may have a gripping element protruding from the wing for engaging with a bone surface. The proximal end of the unthreaded shaft comprises a keyed socket for engaging and rotationally locking with a corresponding key.

The bone implant in an exemplary embodiment of the first aspect may further comprise a threaded lumen concentrically disposed with the keyed socket for engaging with a threaded coupler to lock the key into the keyed socket. The second tunnel may be configured to receive a second screw in a direction inclined relative the longitudinal axis and to the transverse direction of the first screw, wherein the second screw if configured to provide a dynamic compression when installed. At least the unthreaded end-piece may comprise Grade 5 Titanium. It will be appreciated that alternative suitable materials may be used

According to a second aspect there is provided a jig for aligning surgical drills with the bone implant of any of the exemplary arrangements according to the present specification, wherein the jig comprises:

an L-shaped component configured to be coupled to the implant by means of a coupler; one or more tunnels configured to align a hole formed device or to hold an aligning component such as a drill sleeve at a required position and alignment relative to the implant.

According to a third aspect of the specification there is provided a kit for bone fracture or orthopaedic repair surgery, comprising:
a bone implant according to an exemplary arrangement of the present specification; and
a jig according to arrangements of the specification.

According to a fourth aspect, the present specification provides methods for fixation of a fracture in an orthopaedic procedure. In particular, there are provided methods of installing and using a bone implant according to any of the various exemplary embodiments of the specification and optionally using a jig of the present specification in an orthopaedic procedure to provide a system for fixation of a fracture. The system effectively defines a compression and suspension system based on the arrangement of the implant and the bone fixing screws arranged by the method to interconnect and interact with the implant, which together provides for application of and control of forces, by virtue of the arrangement of the implant, to provide effectively fixation of the fracture.

The method comprises:
forming a first hole at the fracture site;
inserting the implant into the hole, wherein the implant is threaded to engage the bone as driven inwardly, wherein a proximal end of the implant has a head which is configured to engage the surface of the bone, and a distal end of the implant is configured to be located distally in the hole, the distal end comprising an archway defining a first tunnel for receiving a first bone fixing screw;
drilling a hole to receive a first bone fixing screw, the hole being drilled to align with the implant such that the hole is formed to receive the screw and to locate it in the first tunnel;
locating the first bone fixing screw in the first tunnel, such that the interaction of the bone fixing screw with the implant at the archway defines a suspension of the implant in the bone through the application of forces including by the bone fixing screw located in the first tunnel at the distal end of the implant and substantially transverse to the implant, and wherein the head at the proximal end engages the bone at the proximal end.

The method may further comprise determining a rotational orientation of the inserted implant about the longitudinal axis thereof, wherein the head is not symmetrical and wherein determining the orientation of the head of the implant allows determination of the rotational orientation of the implant and the location and orientation of the archway and the first tunnel. Similarly, the orientation of the head allows determination of the orientation of the second tunnel.

The method may further comprise forming the first hole for receiving the implant in a direction generally transverse to the fracture. Further the method may comprise forming the holes for receiving the first and if used the second bone fixing screws at an inclination to the longitudinal axis of the implant. The first bone fixing screw may be arranged generally transverse or perpendicular to the implant. The second may be arranged at an inclination to the implant. The inclination may be that the screw is driven at an angle in the range of 30 to 80 degrees to the implant. Alternatively it may be the range of 50 to 70 degrees.

According to the method, a portion of the implant when inserted is located distally of the fracture and a portion is located proximally of the fracture at the surface of the bone.

The first bone fixing screw when arranged in interlocking engagement with the archway defines a suspension screw, based on the forces acting at the implant, bone and screw. Effectively the force acting in a generally upward direction at intersection of the implant and the first bone fixing screw at the distal end provides a neutralisation or balancing of the forces acting in a downward direction at the bone and implant.

The implant may further comprise a second tunnel extending through the implant and configured to receive a second screw, the second tunnel being located between the distal end and the proximal end of the implant and arranged at an angle to the longitudinal axis of the implant, the method further comprising:
drilling a second hole to receive the second screw, wherein the second screw defines a dynamic compression screw, the hole being drilled to align with the implant such that the hole is formed to receive the second screw and to locate it in the second tunnel. The method may further comprise:

inserting the implant into the bone in a direction generally transverse to the fracture such that the threaded portion of the implant engages the bone distally and proximally of the fracture; and
locating the first tunnel distally of the fracture;
inserting the first bone engaging screw in a direction substantially transverse to the longitudinal axis of the implant.

The method may further comprise:
the second tunnel being located distally of the fracture;
   inserting the second screw, defining a dynamic compression screw to locate it in the second tunnel, wherein the implant is configured such that when the second dynamic compression screw is received in the second tunnel it is inclined relative to the longitudinal axis of the implant and engages the bone to apply a dynamic compression force.

The method may further comprise:
coupling a jig to the implant, wherein the jig is an L-shaped piece and is configured with a series of jig tunnels, each jig tunnel configured to align a hole forming device to provide a guide such that the one or more holes are located as required at the implant.

According to a further aspect there is provided a suspension and compression system for fixation of a fracture in a bone in an orthopaedic procedure, the system comprising:
a bone implant according to embodiments of the specification configured to inserted into a hole formed by the surgeon in the fractured bone, the implant having form and dimensions selected according to the form and dimensions of the fractured bone;
at least one bone engaging screw configured for interlocking with the implant at a direction inclined relative to a longitudinal axis of the implant when located in the bone;
   the bone implant comprising:
   a shaft having a threaded portion, a distal end, a proximal end, and a longitudinal axis (L-L), and configured to be inserted into the hole formed in the bone, wherein the shaft is configured to be inserted into the bone and to engage the bone distally and proximally of the fracture;
   wherein the distal end of the implant comprises an archway or eyelet, configured such that when the implant is located in the bone, the archway is configured to receive a bone fixing screw in a direction (T-T) that is inclined to the longitudinal axis (L-L),
   wherein the bone fixing screw when located in the archway is configured to apply a suspension force to the implant, wherein the location of the bone fixing screw in the archway provides for stabilisation of the implant and resistance to pullout;
   wherein the proximal end of the implant comprises a surface engaging head, and when the implant is located in the bone, the surface engaging head is configured to engage the surface of the bone.

The system optionally further comprising a jig, wherein the jig is configured for:
coupling to the bone implant, and
providing alignment of surgical drills with the bone implant;
wherein the jig comprises:
an L-shaped component configured to be coupled to the implant by means of a coupler; one or more tunnels configured to align a hole formed device or to hold an aligning component such as a drill sleeve at a required position and alignment relative to the implant to allow engagement of one or more screws with the implant.

The system optionally further comprising, the implant having a second tunnel for receiving a second screw, the second tunnel screw located near a mid-portion, between the distal and proximal ends, of the implant,
the second screw comprising a dynamic compression screw, the second tunnel configured to receive the dynamic compression screw in a direction that is inclined to the longitudinal axis (L-L) of the implant, wherein the implant is configured such that when the second dynamic compression screw is received in the second tunnel it is inclined relative to the longitudinal axis of the implant and engages to apply a dynamic compression force.

In one arrangement, the first screw may be inserted into and received in the archway in a direction substantially transverse to the longitudinal axis of the implant when the implant located in the bone.

In one arrangement, the implant may be inserted into the bone in a direction that is generally transverse to the fracture to engage the bone distally and proximally relative to the fracture.

In one arrangement, the implant may be located in the bone such that the first tunnel and, if provided in the implant, the second tunnel are both located distally of the fracture.

The scope is in accordance with the appended claims.

### BRIEF DESCRIPTION OF THE DRAWINGS

Embodiments of the present disclosure will now be described, by way of example, with reference to the accompanying drawings, in which:

FIG. 1a shows schematically an ankle with a damaged medial-malleolar;

FIG. 1b shows schematically the ankle of FIG. 1a when fixed using a known fixation;

FIG. 1c shows schematically the ankle of 1a when fixed with an embodiment of the present disclosure.

FIGS. 2a to 2c shows schematically various fixing configurations that are possible with embodiment of the present disclosure;

FIGS. 3a to 3c shows steps in a surgical process to implant embodiment of the present disclosure;

FIGS. 4a and 4b shows steps in the process of using a guiding jig with embodiments of the present disclosure;

FIG. 4c shows an example type bone screw that may be used with embodiments of the present disclosure;

FIGS. 5a and 5b shows two orthogonal side views of a bone implant according to some embodiments of the present disclosure;

FIG. 5c shows a central cross section of the bone implant shown in FIG. 5a;

FIGS. 5d and 5e show end views of the bone implant shown in FIG. 5a;

FIGS. 6a to 6e show views of a bone implant according to some cannulated embodiments of the present disclosure;

FIGS. 7a to 7d shows views of a bone implant according to some cannulated embodiments of the present disclosure;

FIGS. 8a to 8b shows the guiding jig and how the jig may be attached to embodiments of the present disclosure;

FIGS. 9a and 9b show two views of a drill sleeve that may be used with the guiding jig shown in FIG. 8a; and

FIGS. 10a to 10d show images of a system comprising a bone anchor according to some embodiments of the present disclosure mounted on a guiding jig with two drill sleeves;

FIG. 11 is an illustration indicating the exemplary forces. and directions of the forces. arising when a bone implant according to arrangement of the specification is installed for fixation of an ankle fracture.

### DETAILED DESCRIPTION

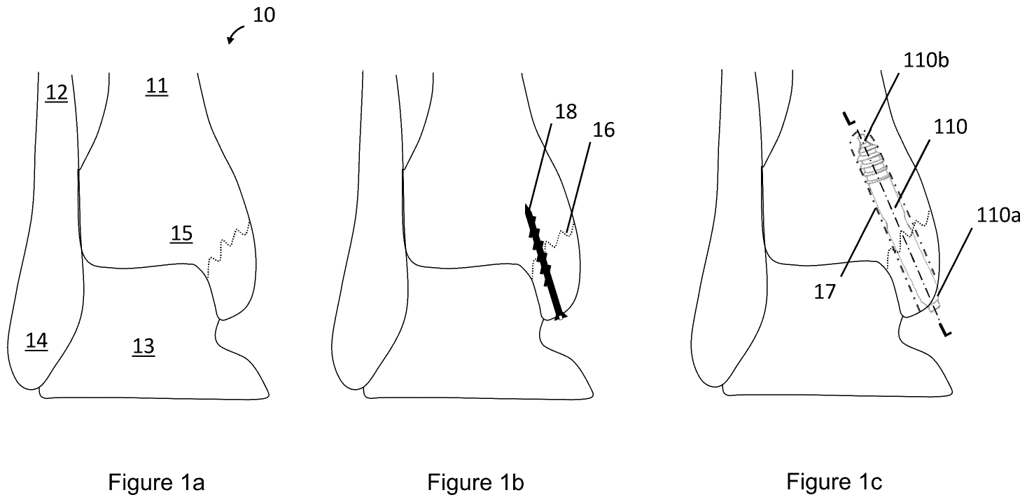

The ankle 10 is a region of the body comprising the complicated joint between a foot and a leg. FIG. 1a show a schematic image of the relevant anatomy. The ankle 10 joins several bones including the ankle bone 13, or talus, located at the top of the foot and the lower leg bones: the tibia 12 and fibula 11. The lower regions of the tibia 12 and the fibula 11 have a bony prominence called the lateral malleolus 14 and the medial malleolus 15 respectively. Both malleoli are part of the mechanical linkage to the talus 13. There are also many other anatomical elements that form and support the ankle 10 including ligaments, tendons, and muscles. A common ankle 10 injury is damage to the medial malleolus 15. This damage can take many forms including fractures. As explained above, often surgery is preferred to support, or fix, the medial malleolus 15. A securely fixed damaged medial malleolus 15 is likely to heal better and there are likely to be less complication during the healing process.

FIG. 1b shows a known way in which a damaged medial malleolus 15 may be repaired. Specifically, it shows a fracture 4 in the medial malleolus 15. Such a fracture 16 can be simple or complex, typically they will significantly reduce mobility of the joint and cause the patient pain. In severe cases, a patient may find it almost impossible to walk. The known way to fix such a damaged medial malleolus 15 involves surgically implanting a screw 18 into the medial malleolus 15. The screw 18 is typically partially threaded and serves to compress medial malleolus 15 in such a way that the fracture 16 is secured, thereby assisting healing. An alternative known method is to fix a plate of stiff material, such as metal fastened by screws, to secure the medial malleolus 15.

Known bone anchor designs operate best when the screws are fastened in strong, or high-quality, bone. Known designs do not operate well in low-quality bone, in which they are often implanted, as low-quality bone is common for many reasons. For example, Osteoporosis, which will affect 1 in 4 women and 1 in 5 men globally, is a bone disease causing lower bone density and therefore much weaker bones that are prone to fracture. Implants are much more likely to fail, e.g., pull out or work loose if they are implanted in osteoporotic bone.

A new bone fixing implant anchor has been designed that can perform well in all bone, including low-quality bone. An embodiment of the new implant 110 is shown in FIG. 1c. The implant 110 defines a longitudinal axis L-L. The implant 110 is typically received into a hole 17 formed in bone. The implant 110 has two ends a distal end 110a that is configured to be inserted furthest into the hole 17 and a proximal end 110b which typically remains just proximal or outside the hole 17. The new implant 110 is twisted into the hole 17 by manipulating it from its distal end 110a, which in use is the end facing the surgeon.

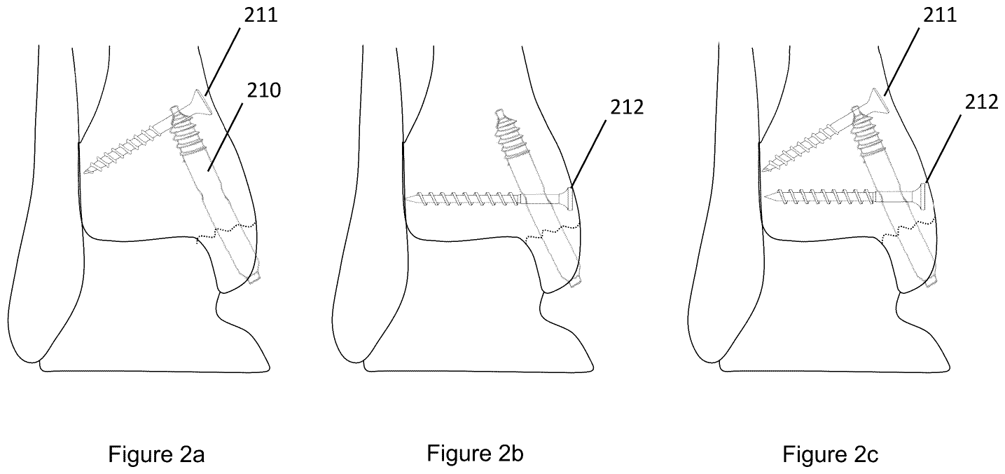

FIGS. 2a to 2c show various fixing configurations that some embodiments of the new implant support. FIG. 2a shows an implant with a screw 211 inserted through the implant 210, at approximately a perpendicular angle and near the distal end of the implant. The screw 211 can be a standard bone screw. It will be appreciated that in the drawings the screws shown are of a simplified form for the purposes of clarity of illustration and to allow illustration of the screws passing through the tunnels and in position relative to the tunnels. It will be appreciated that any suitable bone screw for example a cortical bone screw may be used. A screw in this position is called a suspension screw 211. The suspension screw 211 serves to lock i.e. suspend the implant 210 in position-further securing the medial malleolus 15. The screw 211 as arranged interlocked with the implant defines a suspension screw.

Alternatively or additionally, a screw 212 can be inserted approximately through the implant 210, approximately midway along the longitudinal length of the implant 210. A screw in this position is called a dynamic compression screw 212. The longitudinal axis of the dynamic compression screw 212, when inserted into position, is inclined to the longitudinal axis of the implant 210. The angle of inclination may range from 30 to 90 degrees. The dynamic compression screw 212 serves to lock the implant 210 into position.

The length of screws 211 and 212 is selected according to the location of the fracture and to the bone in which the implant is to be inserted. As shown in FIGS. 2a, 2b, and 2c, the screws selected for interlocking with the implant extend from cortex to cortex. It is noted that the screws are threaded to the distal ends thereof and that they are configured to engage the bone at the thread when inserted distally of the implant. The screws may further comprise a head configured to engage the bone proximally of the implant.

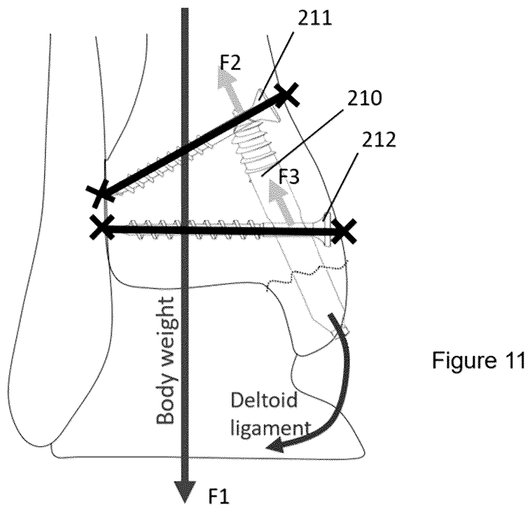

The interactions of the screw 211 or the screws 211 and 212 and the implant 210 and the relevant forces are described further below with reference to exemplary FIG. 11.

The suspension screw 211 and the dynamic compression screw 212 each provide further source of compression to compress the medial malleolus 15. This additional compression serves to further secure the medial malleolus 15. The compressions provided by the dynamic compression screw 212 is particularly helpful to stabilise complex fractures. In other embodiments, additional ancillary screws may be present to help further support the medial malleolus 15 or more firmly secured the implant 310.

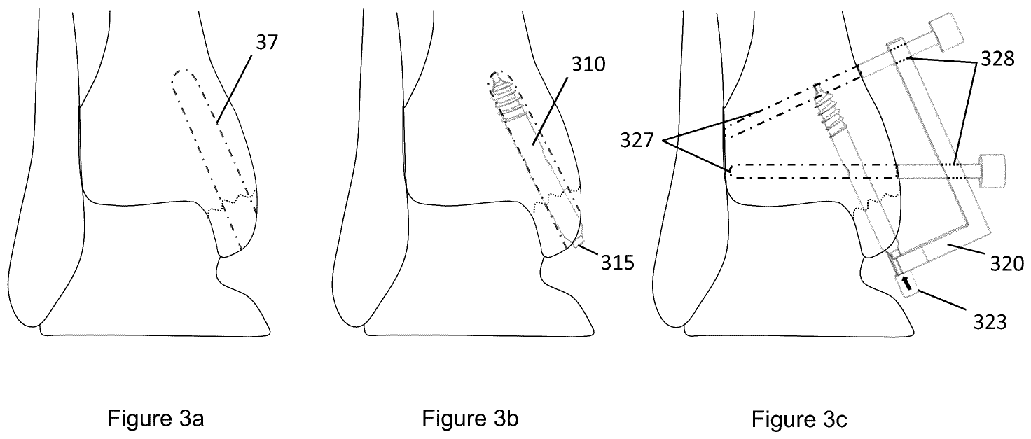

The surgical process by which an implant 310 is typically implanted is shown in FIGS. 3a to 3c. The first step shown in FIG. 3a is to form a hole 37 in the medial malleolus 15. This might be achieved by surgically drilling or any other known hole forming method. The outline of the hole 37 is illustrated with a dotted and dashed line.

As shown in FIG. 3b, the implant 310 is then inserted into this hole 37. As the implant 310 is threaded, the surgeon can drive the implant 310 into the hole by twisting the proximal end of the implant so the thread engages the bone and drive the implant inwardly. The proximal end of the implant has a wide head 315 which extends beyond the hole. The wide head 315 assists in twisting the implant 310 into the hole 37 and prevents the implant going too far into the hole 37. The wide head 315 also engages i.e. cusps the surface of the bone further increasing the stability of the implant 310.

A bone adhesive may also be used to assist in securing the implant 310 into the hole 37. Many known adhesives may be used such as other calcium phosphates or synthetic bone cement. Some of these adhesives may help achieve better augmentation of the implant in low-quality bone and/or may promote bone growth.

FIG. 3c shows the implant 310 once secured in the bone. The hole 37 into which the implant 310 has been inserted is not shown for clarity. The wide head is not cylindrically symmetric and therefore the orientation of the wide head 315 on the surface of the bone can be used to deduce the rotational orientation about the longitudinal axis of the implant 310. Once this is known, the surgeon can drill one or two holes 327 to receive the auxiliary screws, such as the above described suspension screw 211 and/or the dynamic compression screw 212.

The holes 327 for the ancillary screws pass through tunnels in the implant 310. It is therefore important that these are precisely aligned to avoid damage to the implant 310 and/or hole forming tools, and to ensure each hole is sufficiently well formed to safety receive its corresponding ancillary screw. It should be noted that, in some embodiments the tunnels through the implant 310 may allow holes to pass through a range of angles. This eases the requirement on alignment making it easier for surgeons to form the holes but potentially offers a slightly less secure fixing.

To maintain a secure fixing the tunnels can be angled such that they are limited to accepting holes, and according interconnecting screws, within a small or limited range of angles. To assist surgeons in the task of forming holes within these small ranges of angles, the implant 310 can be coupled to a jig 320. The jig 320 is an L-shaped piece designed to be coupled to the implant 310 with a coupler 323. Once coupled, the jig 320 is in fixed mechanical alignment with the implant 310. The jig 320 is configured with a series of tunnels 328. Each tunnel can be used to align hole forming devices e.g. drill, or to hold aligning components e.g. drill sleeves to align hole forming devices. In either case the alignment provided directly or indirectly by the jig 320 ensures the formed one or more holes 327 pass precisely through the implant 310.

In some embodiments the tunnels through the implant 310 may allow holes to pass through a range of angles or multiple discrete angles. In these cases, the jig may have multiple tunnels directed towards the same tunnel in the implant 310. This allows the surgeon to select the appropriate one of the multiple tunnels based on the angle of ancillary screw that the surgeon desires.

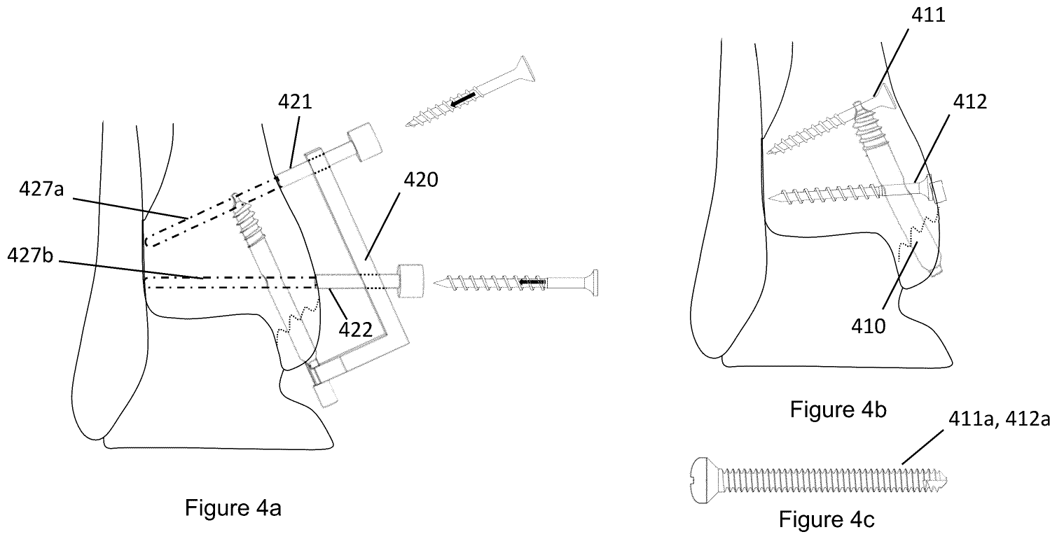

The use of the jig 420 is further illustrated in FIGS. 4a and 4b. The jig 420 and implant 410 have been coupled. In the illustrated jig 420 in FIG. 4a, there are two tunnels that receive each receive a drill sleeve. The first drive sleeve 421 is aligned to assist drilling a suspension screw hole 427a, which passes through near the distal end of the implant, for a suspension screw 411. The first drive sleeve 421 is aligned to assist drilling a dynamic compression screw hole 427b, which passes through the near the middle of the implant in the longitudinal direction, for a dynamic compression screw 412. The suspension screws and dynamic compression screws, such as 411, 412, as illustrated in the figures are simplified for the purposes of clarity of illustration. FIG. 4c illustrates a bone screw 411a, 412a that may be used as suspension and dynamic compression screws. The screw may be a cortical screw. The screw may be of any type suitable for the applications of the present arrangements.

Once both holes 427a, 427b have been formed, the jig 420 is decoupled from the implanted implant 410. The suspension screw 411 is then screwed into the suspension screw hole 427a, and the dynamic compression screw 412 is screwed into the dynamic compression screw. The resultant configuration is shown in FIG. 4b.

Depending on the surgical situation, one of the suspension screw 411 or the dynamic compression screw may not be used. If, for some reason, the formed hole or holes are unsuitable, the jig 420 can be recoupled and the drilling process repeated. As with the implant 410, bone adhesive may also be used to assist in securing either or both of the ancillary screws. The process is the same for implants with more than two ancillary screw tunnels. If the jig 420 has multiple tunnels for an ancillary screw tunnel, the surgeon can use the appropriate tunnel for the angle they require e.g. they can insert the drill sleeve in the appropriate tunnel so that the hole is formed at their preferred angle.

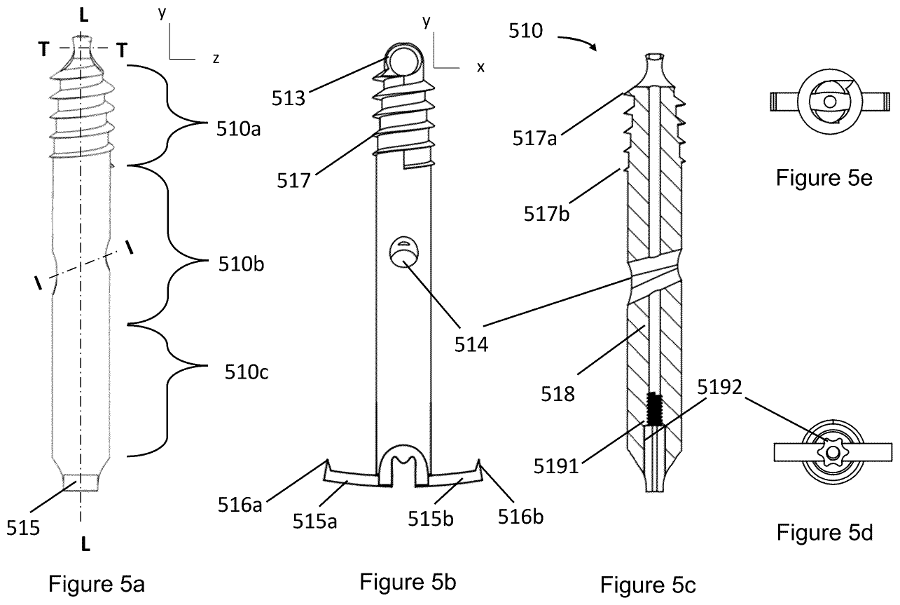

FIGS. 5a to 5d provide more details of some embodiments of the implant 510. FIG. 5a shows the longitudinal axis L-L defined by the substantially cylindrically symmetric body of the implant 510. In the illustrated embodiment the cylindrically symmetric body of the implant 510 can be characterized by three regions: a distal region 510a, central region 510b and proximal region 510c.

Extending from the distal end of the distal region 510a is an archway 513. The archway defines a tunnel, or eyelet, and the tunnel defines a traverse direction T-T. The traverse direction T-T is perpendicular to the longitudinal direction L-L. The tunnel is also configured to receive a standard bone screw (see example FIG. 4c) and defines a diameter of the order of 3.7 mm and 4.5 mm. In a preferred arrangement the diameters may range from between 4.1 and 4.3 mm. The tunnel may be shaped and/or formed with a tolerance to accept a variety of diameter bone screws and/or bone screw with a range of angles from the direction. The eyelet in the exemplary arrangement has a generally cylindrical form, or a circular form in cross-section.

At approximately the middle of the implant 510 in the longitudinal direction L-L, the central region 510b is configured to define a second tunnel 514 through the central region 510b. The second tunnel 514 defines a channel or through hole or aperture. The second tunnel 514 defines an inclined direction I-I. The longitudinal direction L-L, traverse direction T-T, and inclined direction I-I are all in the same plane. The inclined direction I-I is inclined relative to both the longitudinal direction L-L and the traverse direction T-T. The angle of inclination between the inclined direction I-I and the longitudinal direction L-L varies between embodiments but is typically in the range 30 to 80 degrees. In preferred arrangements according to the specification the angle may be in the range of 55 to 75 degrees. In a most preferred arrangement, the angle may be between 60 and 70 degrees. The second hole or the second tunnel 514 may be flared such that the diameter increases or tapers outwardly from a first opening to the second opening The second tunnel 514 may be shaped and/or formed with a tolerance to accept a variety of diameter bone screws and/or bone screw with a range of angles from the direction.

In a preferred embodiments the dimensions of the implant may be varied and selected taking account of typical dimensions and form of the bone in which the implant is to be inserted. Again, the angles of the first and second tunnels of the implant may be varied and selected also taking account of the surgical site and the bone and location in which the implant is to be inserted. It will be appreciated that the form and dimensions may be varied accordingly, and the scope of the present specification covers such variations.

For each embodiment, each tunnel can be matching to bone screw as required by the surgical scenario. For example, if a generous amount of tolerance is allocated to receive a particular bone screw this will lessen the precision needed to pass the particular bone screw through the tunnel. Alternatively, if the tunnel is formed to precisely match a particular bone screw this will result in more secure locking of the implant once the particular bone screw is inserted but will require very precise insertion of the particular bone screw.

The distal region 510a and the central region 510b have approximately similar diameters. However, the distal region 510a has a thread comprising a protruding helical wall forming a thread 517. The thread 517 protrudes radially more and other parts and thereby allows a user to twist, or screw the implant 510 into an appropriately sized hole in bone. The distal end of the distal region may reduce in diameter in a tapered manner in order to increase the protrusion of the distal end of the wall without increasing the overall diameter.

The wall of the thread 517 encircles the distal region 510a at least twice (720 degrees) and preferably 4 to 5 times. The thread 517 is configured to engage with a bone and secure the implant 510 into the bone when it is implanted.

In other embodiments, the wall may by circularly symmetric and/or there may also be multiple walls. Each wall may also be continuous; or formed of several discrete and/or separate parts. Any variation of wall will be usable provided it can firmly secure the implant into bone.

Some embodiments use a wall based on standard thread patterns. However, the profile of the thread 517 can be optimized as shown in FIG. 5c. The optimized thread is specifically designed to work in low-quality bone

The optimization includes variation of the protrusion of the thread 517 along its length. In FIG. 5c, the thread protrusion, or depth, as measured from the surface of the distal region, increases from the (proximal) start of the thread 517a to the (distal) end of the thread 517b. The variation in the protrusion of the thread 517 can help account for the variation in bone quality. For example, when implanted, the distal end of the implant 510 is typically in bone where the quality is likely to be high).

The thread 517 also has an asymmetric profile, or cross-section. The asymmetric profile comprises two profiles: a distal profile and a proximal profile. The asymmetric profile is a type of buttress thread and comprises two profiles with different angles from the longitudinal axis L-L. The distal profile, which is preferably less than 75 degrees and more preferably less than 60 degrees, has a slope that is less inclined than the slope of the proximal profile. The proximal profile is therefore closer to perpendicular with the longitudinal axis L-L than the distal profile. The asymmetric thread profile therefore increases the compression force that the implant can apply relative to standard designs.

To explain the operation of this asymmetric profile, the implant 510 is inserted into the bone by applying a force in the distal direction and a twisting insertion motion in a clockwise direction (looking from the proximal end around the longitudinal axis L-L). During insertion, the thread 512 cuts or deforms the bone that it engages with to form a thread path in the bone. The engagement of the thread 517 with the formed thread path secures the implant 510 in the bone. The insertion process is like screwing a wood screw into wood. Once engaged, or screwed-in, the flatter proximal profile of the asymmetric profile provides a strong grip allowing a large distal/compression force to be applied.

The diameters of each portion or region of the implant are selected including such that the second tunnel 514 can be sufficiently sized to receive standard bone screws, without the second tunnel adversely affecting the structural integrity of the central region 510b.

In an exemplary arrangement, the central region or midshaft 510b may be modified in form and dimension. In particular, the region between the tunnel 514 and extending upwardly to the threaded shaft portion may be relatively thickened (increased diameter and radius in the lateral cross-section direction) in order to increase strength taking account of the reduced thickness of the wall of the implant at the tunnel 514.

The head 515 of the implant is at the proximal end of the proximal region 510c. It is called the head 515 only because once the implant 510 is inserted into the formed drill it remains outside the hole i.e. proud of the bone surface. The head 515 is wide in that it extends in the radial direction (perpendicular to the longitudinal direction L-L more than any other part of the implant 510. As explained above, the wide nature of the head 515 serves to engage i.e. to cusp the surface of the bone when the implant is secured in the formed hole.

In the arrangements of the specification as noted above the bone screw engaged with the distal end tunnel or archway effectively provides suspension forces. The bone screw engaged with the centrally located tunnel is active in providing compression forces. The head which is configured to cusp the surface of the bone at the proximal end is also effectively active in providing compression forces.

The embodiment shown in FIGS. 5a to 5e shows a particular embodiment in which the head 515 is formed by two substantially parallel, radially extending, protrusions 515a, 515b on opposing sides of the implant. These protrusions 515a, 515b, form a narrow strip. By limiting the head to essentially a narrow strip the surface of the bone in contact with the head is relatively limited, making it easier for the body to heal around the implant. The narrow strip also serves to indicate the rotational orientation of the implant 510 around the longitudinal axis L-L. This rotational orientation defines the angle of the tunnels. Therefore, the narrow strip can indicate the direction in which the hole will have to be formed, which avoids holes having to be formed at awkward angles.

At the radial edge of either protrusion, is a spike 516a, 516b that is configured to project to the surface of the bone outside the formed hole, as described above to cusp the bone at the surface. The spikes 516a, 516b rotationally lock at the bone surface and help further secure the implant.

The protrusions 515a, 515b are inclined slightly distally to help locate the spikes at the bone surface. The spikes are configured to cusp the bone at the surface. In some examples, the protrusions can be curved. In one preferred example, the protrusion are 1 mm thick in the longitudinal direction, 15 mm across in the radial direction, 2.5 mm deep, and formed to define a 45 mm circle centred on the eyelet.

In the proximal region 510c, there is a jig connection 5191 for connecting the implant 510 to an insertion jig. The coupling connection can be anything that can couple the implant 510 to the insertion jig. In one preferred example, the jig connection is a threaded hole.

At the proximal end of the proximal region 510c is a connection 5192. This can be used for coupling to a driving tool to drive or twist the implant 510 into bone. The connection 5192 allows a surgeon to use a driving tool to insert and/or retract the implant 510. Multiple forms of keyed connections are possible including machined receptacles or protrusions for coupling to matching tools e.g., hex slots and hex drivers; features to facilitate gripping such as knurling; and magnetic coupling. Any feature that allows a surgeon to forcibly manipulate the implant 510 from the proximal end can be used as a connection. In the preferred example shown in FIG. 5c, the connection 5192 is a six-point star shaped socket. Such a socket helps ensure the implant 510 is well aligned with the driving tool before the implant 510 is twisted into place.

Referring to FIG. 5c, the central axis of the implant 510 comprises a channel or bore 518 that runs between openings at the distal and proximal ends. In the arrangement shown the implant comprises a bore 518 from distal to proximal end having a threaded jig connection 5191 at the proximal end. The channel or bore of the implant effectively defines a cannulated implant. The cannulated implant is configured to receive for example a guide wire.

Referring to FIGS. 5d and 5e, it is illustrated that the orientation of the head 515, 516 is keyed to that orientation of the archway 513 and tunnel 514. During use when the implant is inserted into the bone, the orientation of the head provides an indication to the surgeon of the orientation of the first and second tunnels for alignment with interlocking screws.

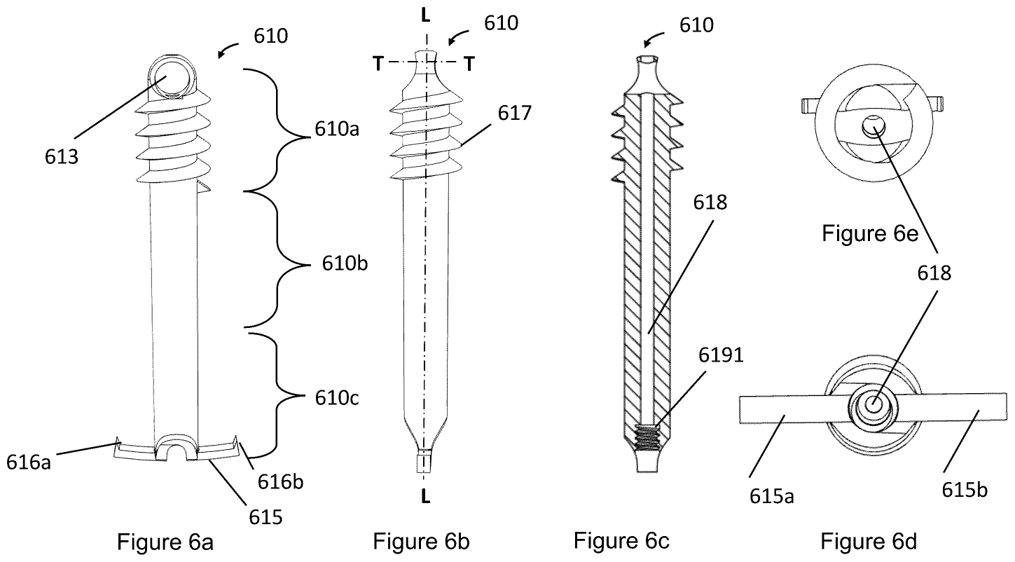

Referring to FIGS. 6a to 6e an implant 610 according to a further exemplary arrangement of the specification, is described. The implant 610 is similar to the implant 510 and similar reference numerals have been applied as appropriate. However, the implant 610 comprises a cannulated implant having a channel or bore 618 extending along the central longitudinal axis of the implant in the direction (L-L). The channel 618 to define cannulated implant and the cannulated form if configured to provides for an additional visual aid for the surgeon, including for guidewire insertion and guidance through surgical process.

The implant comprises an archway 613 at the distal end and a head 615 at the proximal end and having a longitudinal axis arranged in the direction (L-L). The archway defines an aperture or channel or tunnel. The tunnel is configured to receive a bone fixing screw 212 at the archway. The tunnel is oriented in a lateral direction across the body of the implant. The tunnel is arranged in a direction (T-T) substantially transverse to the direction of the longitudinal axis of the implant.

The distal region 610a and the central region 610b have approximately similar diameters. However, the distal region 610a has a thread comprising a protruding helical wall forming a thread 617. The thread 617 protrudes radially more and other parts and thereby allows a user to twist, or screw the implant 610 into an appropriately sized hole in bone. The distal end of a proximal region 610c may reduce in diameter in a tapered manner in order to increase the protrusion of the distal end of the wall without increasing the overall diameter.

The embodiment shown in FIGS. 6a to 6e shows a particular embodiment in which the head 615 is formed by two substantially parallel, radially extending, protrusions 615a, 615b on opposing sides of the implant. These protrusions 615a, 615b, form a narrow strip. By limiting the head to essentially a narrow strip the surface of the bone in contact with the head is relatively limited, making it easier for the body to heal around the implant. The narrow strip also serves to indicate the rotational orientation of the implant 610 around the longitudinal axis L-L. This rotational orientation defines the angle of the tunnels. Therefore, the narrow strip can indicate the direction in which the hole will have to be formed, which avoids holes having to be formed at awkward angles.

At the radial edge of either protrusion, is a spike 616a, 616b that is configured to project into the surface of the bone outside the formed hole. The spikes 616a, 616b rotationally lock at the bone surface and help further secure the implant. The spikes are configured to cusp the bone surface.

The protrusions 615a, 615b are inclined slightly distally to help drive the spikes to locate the spikes so as to cusp the bone surface. In some examples, the protrusions can be curved. In one preferred example, the protrusions are 1 mm thick in the longitudinal direction, 15 mm across in the radial direction, 2.5 mm deep, and formed to define a 45 mm circle centred on the eyelet.

In the proximal region 610c, there is a jig connection 6191 for connecting the implant 610 to an insertion jig. The coupling connection can be anything that can couple the implant 610 to the insertion jig. In one preferred example, the jig connection is a threaded hole. The inner portion of the implant for receiving the insertion jig may comprise a thread to allow the insertion jig to be connected.

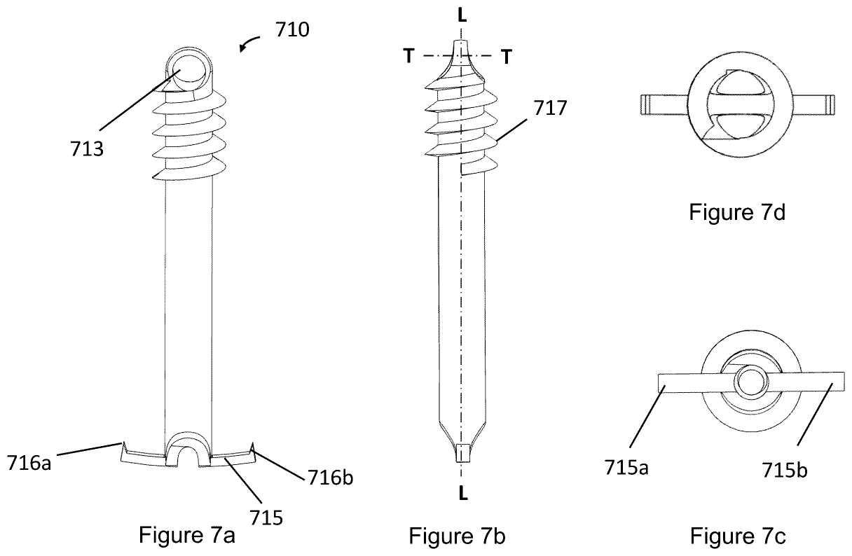

Referring to FIGS. 7a to 7d an implant 710 of an alternative arrangement is shown. The implant 710 is similar to the implants 510 and 610 and similar reference numerals have been applied as appropriate. However, the implant 710 comprises an archway 713 at a distal end and a head 715, 716 at a proximal end and having a longitudinal axis arranged in the direction (L-L). The features of the archway and head are similar to those described above with reference to FIGS. 5 and 6. The archway defines an aperture or channel or tunnel. The tunnel is configured to receive a bone fixing screw 212 at the archway. The tunnel is oriented in a lateral direction across the body of the implant. The tunnel is arranged in a direction (T-T) substantially transverse to the direction of the longitudinal axis of the implant.

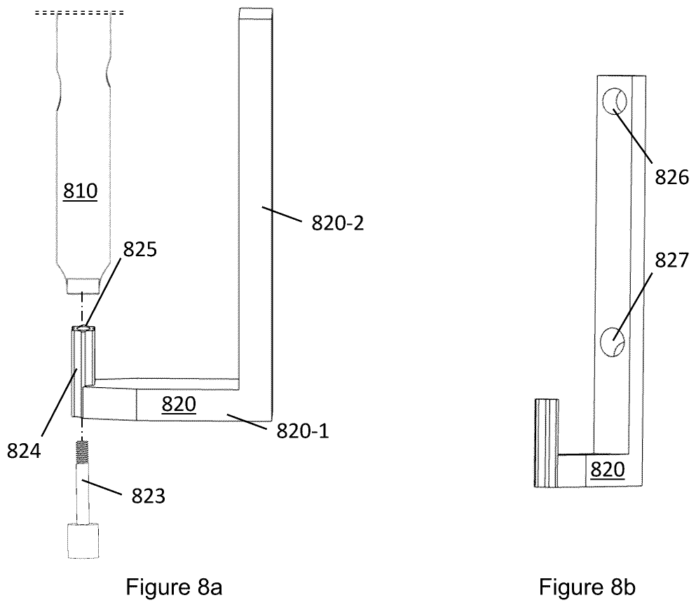

Referring to FIGS. 8a, 8b, 9a, 9b, 10a-d jig arrangements 820 and 1020 for aligning surgical drills with the bone implants 110, 210, 310, 510, 610, 710, 810 and 1010, according to arrangement of the specification are described. The jigs 820 and 1020 are similar to the jig 420 of FIG. 4a and the description provided applies also to these drawings.

In each case, the implant may comprise a receiver and inner threaded portion for receiving the insertion jig may comprise a thread to allow the insertion jig to be connected.

The jig 820 comprises an L-shaped component having a first arm 820-1 and a second arm 820-2 at right angles to each other. The jig 820 is configured to be coupled to implant 810 according to the exemplary arrangements of the specification by means of a coupler 823 located at a protruding engagement portion 824, 825 of the jig receivable at the proximal end of an implant 810. One or more tunnels 826 and 827 are provided configured to align a hole formed device or to hold an aligning component such as a screw sleeve and/or drill sleeve at a required position and alignment relative to the implant.

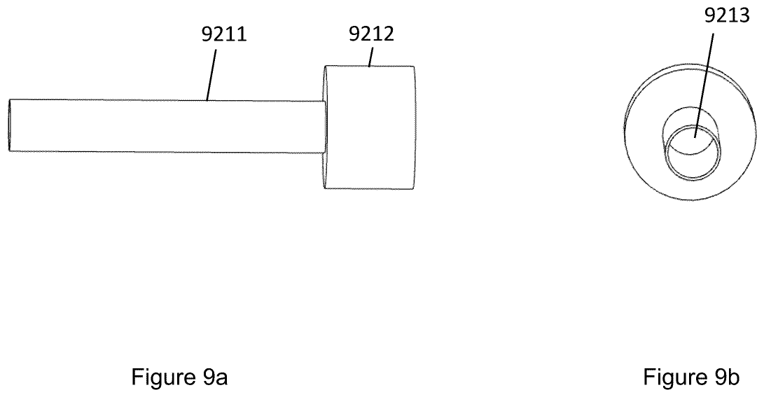

Referring to FIGS. 9a and 9b drill sleeves 9211, 9212 having a drill channel 9213 are illustrated. The sleeves illustrated in FIGS. 9a and 9b can also be screw sleeves. Drill sleeves may be used together with the jigs of the present specification to provide for alignment for drilling to insert the one or more interlocking screws into an implant in a bone. Screw sleeves may be used together with the jigs of the present specification to provide for alignment for drilling to insert the one or more interlocking screws into an implant in a bone.

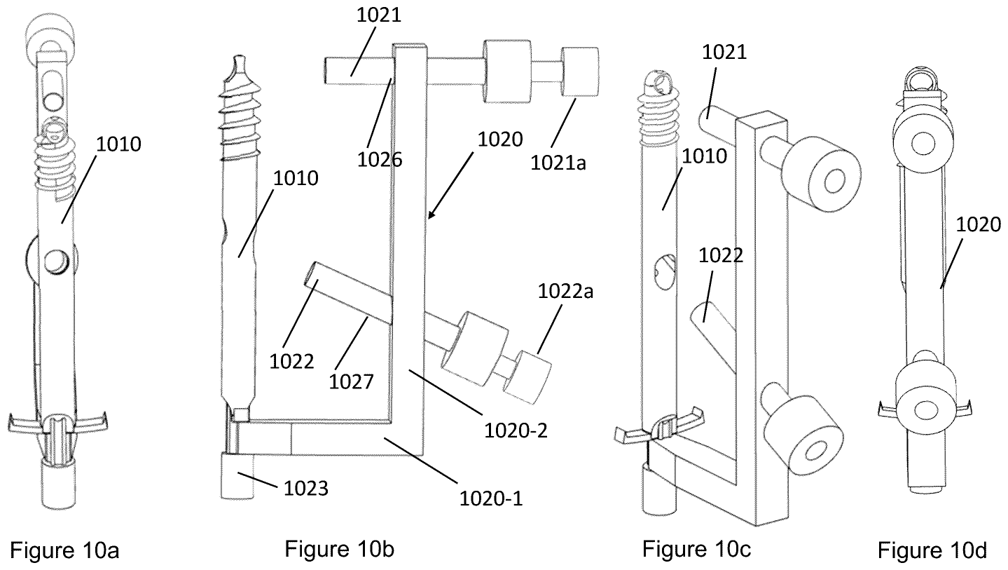

Similarly, to the jig 820 (and the jig 420), the jig 1020 comprises an L-shaped component having a first arm 1020-1 and a second arm 1020-1 at right angles to each other. The jig 1020 is configured to be coupled to implant 1010 according to the exemplary arrangements of the specification by means of a coupler 1023 located at a protruding engagement portion (not shown in FIGS. 10a-d, but similar to engagement portion 824, 825) of the jig receivable at the proximal end of an implant 1010. One or more tunnels 1026 and 1027 are provided configured to align a hole formed device or to hold an aligning component such as a drill sleeve at a required position and alignment relative to the implant. In the arrangement of FIGS. 10a-d, screw sleeves 1021, and 1022 are shown located at the tunnels 1026 and 1027 of the jig 1020. Drill sleeves may be attached to the screw sleeves 1021, 1022. In the arrangement of FIG. 10b, drill sleeves 1021a and 1022a are shown located at a distal end of the screw sleeves 1021, 1022.

It will be appreciated that the form and dimensions of implants 110, 210, 310, 410, 510, 610, 710 according to the exemplary arrangements of the specification may be varied as required including taking account of the location of the fracture to be fixed. According to the arrangements of the specification a range of implants of different length may be provided. Typical exemplary lengths are within the range of 40 to 80 mm for example. In exemplary preferred arrangements implants having a length in the range of 45 to 55 mmm may be provided. In preferred exemplary arrangements the distance between the first tunnel at the eyelet hole or archway and the second tunnel defining the compression hole will not change with the changing total length of the implant. In preferred arrangements this distance is of the order of 25 mm. If the diameter of the implant is changed then the diameter of the tunnels may be changed. In a preferred arrangement the tunnels may be of diameter in the range of 3.7 mm to 4.5 mm. In a preferred arrangement the diameter may range from 4.1 mm to 4.3 mm. The first tunnel at the eyelet or archway may be arranged substantially transverse to the longitudinal axis of the implant. The diameter of the implant of a preferred arrangement maybe of the order of 5 mm to 8 mm depending for example on where in the patient the implant is to be implanted. The diameter may vary at portions of the implant from the distal end to the proximal end for example to accommodate the thread at the distal end. Alternatively, the diameter of the body of the implant may be generally constant from distal end to proximal end thereof between the distal archway and the proximal end receiver for connection to a jig. As described the implant may be provided as a cannulated or non-cannulated implant. As an example, it is noted that the cannula or channel may have diameter of the order of 1 to 2 mm, in a preferred exemplary arrangement of the order of 1.5 mm.

Referring to FIG. 11, the forces acting at the fracture site and relating to the implant are described. As discussed, the combination of forces by virtue of the arrangement of the implant and the interlocking screw or screws provide for an improved fixation at the fracture site. In the exemplary arrangement of FIG. 11, the implant is shown as in previous drawings as inserted to providing fixation of an ankle fracture. Force 1 (F1) is proportionate to the % body weight (% body weight). With the assumption that the boundary conditions are fixed-Force 2 (F2) and Force 3 (F3) are proportionate to bending resistance of the screw. F1=F2+F3. Force 2 and force 3 (where suspension/compression screws intersect with main screw (described also in the specification as the implant) namely the suspension mechanism) neutralise Force 1 (relating to the body weight). In the specification, the terms suspension or suspending and compression have been used when describing the interaction relating to the implant and associated one or more interconnecting screws intersect with the main implant. These terms relate to the forces in action as illustrated in FIG. 11. The terms referring to suspension relate to the balancing or neutralising of the Force that are applicable at the implant and intersecting screws.

With reference to the arrangement of the implant when implanted in located it will be appreciated that the interaction of the anchor to cusp the bone at the proximal end may also contribute to the compression forces.

Instead of basing fracture fixation on screw purchase to bone, in the arrangements of the system and device of the disclosure it is based on the bending resistance of the screw. Effectively the arrangements of the device and system of the specification when installed, are bone independent. This provides advantages including in addressing problems associated with previous arrangements—as described further below including pull out resistance and excellent stability.

The surface of the implant may be further treated to improve the bonding by surface treatment. For example, the implant may be etched; textured; plasma treated; anodized, preferably with type II (often sulphuric acid based) anodizing; polished; cleaned; or coated with an intermediate thin film. This treatment may be limited to parts of the implant. For example, the non-threaded parts may be treated to improve bonding to bone adhesive. In other cases, for ease of production, the whole implant is treated the same.

The material of the implant may be selected to take into account factors such as mechanical properties, biocompatibility, and machinability. Typically, the selected material will be a medical grade surgical implant material. For example, the whole implant or part thereof may be formed from, or comprise, at least one of Titanium, PEEK, stainless steel, cobalt chrome. For some embodiments, the implant will be formed from multiple materials to optimise the material properties of individual parts. In one example, the implant is formed from Titanium Grade 5 (Ti6Al4V) because this material provides good mechanical and biological properties, and bonds well with bone adhesive.

The implant is formed to avoid sharp edges, e.g., smoothing, using fillets, or polishing edges afterwards. The threads may also be smoothed into the body of the implant. This helps limit corners and cavities for infection, and reduces the likelihood of damage to a strand of suture located in or near the various features.

Whilst the exemplary arrangements illustrate the present disclosure by reference to ankle surgery, and the exemplary arrangements relate to ankle surgery. It will be appreciated that the present disclosure is not limited to this use.

Bone implants according to the arrangement of the present specification can be used in any situation where a strong fixing to bone is required. In particular, the bone implant may be used in surgical operations for example at the shoulder, knee, elbow, wrist, or hip. Including at the following locations: Medial malleolar, Elbow olecranon fractures, Shoulder greater tuberosity fractures, and Calcaneal tuberosity fracture. It will be appreciated that the form and/or dimension of the implant may be selected or varied as appropriate based on the facture location.

Overall, the arrangements of the specification have a number of advantages including the following:

The implant is configured for and intended to be used in combination with a standard threaded screw and/or compression screw to create a ‘Compression suspension system’. The arrangement and the combination of features of the exemplary embodiments aim to maximise the pullout strength of the implant and also to provides a reduction in infection rates. Further the implant by virtue of the arrangement and system and method provided allows for an increase early weight-bearing in all-age fractures including for example medial malleolar ankle fractures thereby supporting improved outcomes and reduction in risk of future arthritis and total ankle replacement.

Referring to the exemplary arrangements relating to an implant, system and method for ankle fixation of complex medial malleolar fracture with screw-suspension eyelet system modified with a compression hole for extra stability-further aims of the arrangement and advantages brought about by the special combination of features include the following:
maximise pullout strength (resistance to pullout) in osteoporotic bone and healthy bone, thus avoiding failure and supporting a reduction in infection rates and pain after surgery;
configured to be used as a standard and dedicated medial malleolar ankle fixation device;
configured to allow for earlier weight-bearing;
configured to increase stability in low-quality bone.

The arrangement of the specification which provides an archway or eyelet at the distal end of implant (as described above) and configured to receive a standard screw to suspend therethrough provides an improved arrangement for stabilising a fracture for example such as a medial malleolar fracture and improved resistance to pullout. The suspension provided by locating a bone fixing screw through the archway as described advantageously provides for maximising stability of the system. As described above, and with reference to FIG. 11 the systems according to the specification including an implant, and first, or first and second interlocking screws, when applied or installed during surgery at a fracture, as described, are effective including by application of the combination of forces acting together at the implant and the facture with synergy to provide a improved and effective fixation of the fracture.

Arrangements of the present specification may be used for standard type ankle fixations of the medial malleolar (i.e not complex fractures), in particular for such applications the compression-suspension system in either non-cannulated or cannulated form may be provided. The non-cannulated and cannulated forms differ and are provided including to address preferences of the surgeon. As described above the cannulated form provides an additional visual aid for the surgeon, including for guidewire insertion and guidance through surgical process.

When the required application relates to fixation of complex fractures, for example, a complex medial malleolar fracture, the arrangement of the implant including a second tunnel located near the midshaft is preferred allowing for the implementation of a dynamic compression screw located when installed through the second tunnel. The second tunnel is located below the thread to receive the compression screw. The provision of the compression tunnel or compression through hole—for receiving a compression screw provides extra stability and increased security of fracture fixation in complex fractures.

Overall, the arrangements of the implant, the system and methods of the specification based on the implant device and the compression-suspension system supports a new approach in surgery to increase pullout strength and thus minimising implant failure.

1. A bone implant for supporting a damaged bone or for fixing a bone fracture, comprising:
a shaft having a proximal end and a distal end and a longitudinal axis;
the shaft having a threaded shaft portion extending in a proximal to distal direction and located proximate the distal end;
the shaft having an unthreaded shaft portion located between a proximal end of the shaft and the threaded shaft portion;
an archway extending from the distal end of the threaded shaft portion, the archway forming a tunnel that is configured to receive a bone fixing screw in a direction that is inclined to the longitudinal axis; and
the proximal end of the shaft configured to allow a user to drive the shaft into bone.

2. The bone implant of claim 1 comprising a connector at the proximal end of the shaft configured for coupling to a driving tool to drive or twist the bone implant into bone.

3. The bone implant of claim 1, wherein the direction is perpendicular to the longitudinal axis.

4. The bone implant of claim 1, wherein the shaft comprises a second tunnel extending across one of the threaded or the unthreaded shaft portion in a second direction that is inclined to the longitudinal axis.

5. The bone implant of claim 1, wherein a bone fixation implant is a cannulated bone fixation implant and comprises a lumen in a longitudinal direction for coupling to a guidewire.

6. The bone implant of claim 1, wherein the unthreaded shaft portion comprises a proximal part and a distal part.

7. The bone implant of claim 5, wherein a second tunnel is disposed in a distal part.

8. The bone implant of claim 1, wherein a driver comprises two or more wings, the two or more wings extending in a radial direction away from the longitudinal axis and symmetrically arranged about the longitudinal axis.

9. The bone implant of claim 8, wherein a radial tip of each wing has a gripping element protruding from the wing for engaging with a bone surface.

10. The bone implant of claim 1, wherein the proximal end of the unthreaded shaft portion comprises a keyed socket for engaging and rotationally locking with a corresponding key.

11. The bone implant of claim 10, further comprising a threaded lumen concentrically disposed with the keyed socket for engaging with a threaded coupler to lock the key into the keyed socket.

12. The bone implant of claim 4, wherein the second tunnel is configured to receive a second screw in a direction inclined relative the longitudinal axis and to a transverse direction of a first screw, wherein the second screw if is configured to provide a dynamic compression.

13. The bone implant of claim 1, wherein at least an unthreaded end-piece comprises Grade 5 Titanium.

14. (canceled)

15. A kit for bone fracture or orthopaedic repair surgery, comprising:
the bone implant of claim 1; and
a jig configured for use with the bone implant to align surgical drills with the bone implant, the jig comprising:
an L-shaped component configured to be coupled to the bone implant by means of a coupler;
one or more tunnels configured to align a hole formed device or to hold an aligning component such as a drill sleeve at a required position and alignment relative to the bone implant.

16. A method of using a bone implant of claim 1 in an orthopaedic procedure to provide a compression and suspension system for fixation of a fracture, the method comprising:
forming a first hole at the fracture site;
inserting the bone implant into the hole, wherein the bone implant is threaded to engage a bone as driven inwardly, wherein a proximal end of the bone implant has a head which is configured to engage a surface of the bone, and a distal end of the bone implant is configured to be located distally in the hole, the distal end comprising an archway defining a first tunnel for receiving a first bone fixing screw;
determining a rotational orientation of the bone implant about a longitudinal axis thereof, wherein the head is not symmetrical and wherein determining the rotational orientation of the head of the bone implant allows determination of the rotational orientation of the bone implant and a location and orientation of the archway and the first tunnel;
drilling a hole to receive a first bone fixing screw, the hole being drilled to align with the bone implant such that the hole is formed to receive the screw and to locate it in the first tunnel;
locating the first bone fixing screw in the first tunnel, such that the interaction of the bone fixing screw with the bone implant at the archway defines a suspension of the bone implant in the bone through the application of forces including by the bone fixing screw located in the first tunnel at the distal end of the bone implant and substantially transverse to the bone implant, and wherein the head at the proximal end engages the bone at the proximal end.

17. The method of claim 16 wherein the first bone fixing screw when arranged in interlocking engagement with the archway defines a suspension screw, based on the forces acting at the bone implant, bone, and screw.

18. The method of claim 16, the bone implant further comprising a second tunnel extending through the bone implant and configured to receive a second screw, the second tunnel located between the distal end and the proximal end of the bone implant and arranged at an angle to the longitudinal axis of the bone implant, the method further comprising:
drilling a second hole to receive the second screw, wherein the second screw defines a dynamic compression screw, the hole being drilled to align with the bone implant such that the hole is formed to receive the second screw and to locate it in the second tunnel.

19. The method of claim 16, further comprising:
inserting the bone implant into the bone in a direction generally transverse to the fracture such that a threaded portion of the bone implant engages the bone distally and proximally of the fracture;
locating the first tunnel distally of the fracture; and
inserting the first bone engaging screw in a direction substantially transverse to the longitudinal axis of the bone implant.

20. The method of claim 18- or 19, further comprising:
the second tunnel being located distally of the fracture;
inserting the second screw, defining a dynamic compression screw to locate it in the second tunnel, wherein the bone implant is configured such that when a second dynamic compression screw is received in the second tunnel it is inclined relative to the longitudinal axis of the bone implant and engages the bone to apply a dynamic compression force.

21. The method of claim 16 further comprising:
coupling a jig to the bone implant, wherein the jig is an L-shaped piece and is configured with a series of jig tunnels, each jig tunnel configured to align a hole forming device to provide a guide such that the one or more holes are located as required at the bone implant.

22. A suspension and compression system for fixation of a fracture in a bone in an orthopaedic procedure, the system comprising:
a bone implant as claimed in claims 1;
at least one bone engaging screw configured for interlocking with the bone implant at a direction inclined relative to a longitudinal axis of the bone implant when located in the bone.

23. The system of claim 22, further comprising a jig, wherein the jig is configured for:
coupling to the bone implant, and
providing alignment of surgical drills with the bone implant;
wherein the jig comprises:
an L-shaped component configured to be coupled to the bone implant by means of a coupler;
one or more tunnels configured to align a hole formed device or to hold an aligning component such as a drill sleeve at a required position and alignment relative to the bone implant to allow engagement of one or more screws with the bone implant.

24-27. (canceled)
